(19) United States
(12) Patent Application Publication
(10) Pub. No.: US 2023/0149361 A1
(43) Pub. Date: May 18, 2023

(54) METHODS OF TREATING CORONAVIRUS INFECTIONS USING ANGIOTENSIN-CONVERTING ENZYME 2 INHIBITORS

(71) Applicant: Kumuda Inc., Charlestown, MA (US)

(72) Inventor: Parisa Razaz, London (GB)

(73) Assignee: Kumuda Inc., Charlestown, MA (US)

(21) Appl. No.: 17/913,185

(22) PCT Filed: Mar. 29, 2021
(86) PCT No.: PCT/US2021/024685

§ 371 Date: Sep. 21, 2022

Related U.S. Application Data
(60) Provisional application No. 63/004,809, filed on Apr. 3, 2020.
(60) Provisional application No. 63/101,207, filed on Mar. 29, 2020.

(51) Int. Cl. A61K 31/4174; A61K 31/352; A61K 45/06

(52) U.S. Cl. CPC A61K 31/4174; A61K 31/352; A61K 45/06

(57) ABSTRACT

The present invention is directed to, in part, methods of preventing and/or treating coronavirus infection. In some embodiments, provided herein are methods of preventing or treating an infectious disease caused by coronavirus (e.g., SARS, MERS, and COVID-19) comprising administering to a subject in need thereof a compound described herein (e.g., MLN-4760, DX600, ORE-1001, Equivir, Landefill, and other ACE2 inhibitors).

### BACKGROUND

Coronaviruses, such as SARS severe acute respiratory syndrome coronavirus (SARS-CoV), Middle East respiratory syndrome coronavirus (MERS-CoV), and SARS-coronavirus 2 (SARS-CoV-2) can cause respiratory tract infections in human that are lethal. SARS-CoV-2, for example, is believed to be the etiologic agent of the new lung disease coronavirus disease 19 (COVID-19).

Cell entry of coronaviruses depends on binding of the viral spike (S) proteins to cellular receptors and on S protein priming by host cell proteases. SARS-CoV and SARS-CoV-2 utilize angiotensin-converting enzyme 2 (ACE2) as receptor for viral cell entry. Accordingly, ACE2 inhibitors may be useful in treating coronavirus infections.

### SUMMARY

Described herein are methods of treating coronavirus infections. In some embodiments, methods of treating infectious diseases caused by coronaviruses (e.g., severe acute respiratory syndrome (SARS), Middle East respiratory syndrome (MERS), and coronavirus disease 19 (COVID-19)) are provided.

In some embodiments, the present disclosure provides a method of treating infectious diseases caused by coronaviruses in a subject in need thereof, comprising administering to the subject a therapeutically effective amount of an angiotensin-converting enzyme 2 (ACE2) inhibitor.

In some embodiments, the present disclosure provides a method of treating or preventing infectious diseases caused by coronaviruses in a subject in need thereof, comprising administering to the subject a therapeutically effective amount of an angiotensin-converting enzyme 2 (ACE2) inhibitor, wherein the infectious diseases is selected from SARS, MERS, and COVID-19, and wherein the ACE2 inhibitor is selected from MLN-4760, DX600, ORE-1001, Equivir, and Landefill, and pharmaceutically acceptable salts thereof.

In some embodiments, the present disclosure provides a method of treating or preventing SARS or COVID-19 in a subject in need thereof, comprising administering to the subject a therapeutically effective amount of an angiotensin-converting enzyme 2 (ACE2) inhibitor, wherein the ACE2 inhibitor is selected from MLN-4760, DX600, ORE-1001, Equivir, and Landefill, and pharmaceutically acceptable salts thereof.

In some embodiments, the present disclosure provides a method of treating or preventing COVID-19 in a subject in need thereof, comprising administering to the subject a therapeutically effective amount of an angiotensin-converting enzyme 2 (ACE2) inhibitor, wherein the ACE2 inhibitor is ORE-1001, or a pharmaceutically acceptable salt thereof.

In some embodiments, the present disclosure provides a method of treating or preventing COVID-19 in a subject in need thereof, comprising administering to the subject a therapeutically effective amount of an angiotensin-converting enzyme 2 (ACE2) inhibitor, wherein the ACE2 inhibitor is (S,S)-2-[1-carboxy-2-[3-(3,5-dichlorobenzyl)-3H-imidazol-4-yl]-ethylaminol-4-methylpentanoic acid, or a pharmaceutically acceptable salt thereof.

In some embodiments, the present disclosure provides a method of treating or preventing COVID-19 in a subject in need thereof, comprising administering to the subject a therapeutically effective amount of an angiotensin-converting enzyme 2 (ACE2) inhibitor, wherein the ACE2 inhibitor is

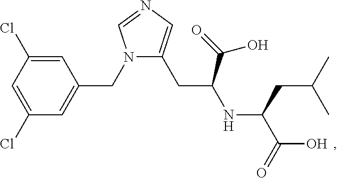 [SMILES: CC(C)C[C@H](N[C@@H](Cc1cncn1Cc1cc(Cl)cc(Cl)c1)C(=O)O)C(=O)O]

or a pharmaceutically acceptable salt thereof.

In some embodiments, present disclosure provides a method of inhibiting transmission of a coronavirus, a method of inhibiting coronavirus replication, a method of minimizing expression of coronavirus viral proteins, or a method of inhibiting coronavirus release, comprising administering a therapeutically effective amount of an ACE2 inhibitor (e.g., MLN-4760, DX600, and ORE-1001), or a pharmaceutically acceptable salt thereof, to a patient suffering from the virus, and/or contacting an effective amount of an ACE2 inhibitor (e.g., MLN-4760, DX600, and ORE-1001), or a pharmaceutically acceptable salt thereof, with an infected cell. Embodiments of the ACE2 inhibitor (e.g., MLN-4760, DX600, and ORE-1001), or a pharmaceutically acceptable salt thereof obstruct viral binding, reduce viral load and downstream effects.

Other objects and advantages will become apparent to those skilled in the art from a consideration of the ensuing Detailed Description, Examples, and Claims.

### BRIEF DESCRIPTION OF THE DRAWINGS

FIG. 1 is an illustration showing a superposition between ACE2 structures, one with ORE-1001 (PDB ID: 1R4L; green), and one with a SARS-CoV-2 spike construct (PDB ID: 7DMU, red [ACE2] and black [spike]).

FIG. 2 is an illustration showing a spike appearing in this case to interact with two moderately flexible helices, on the side of ACE2 to which the leucine of ORE-1001 directs towards.

FIG. 3 is an illustration displaying the molecular surface of the receptor colored by lipophilicity within 15 Å of the ligand, to illustrate a z-clipped look at the ligand site.

FIG. 4 is a depiction of an adapted route to allow installing an appendage.

### DETAILED DESCRIPTION

As generally described herein, the present invention provides methods for treating infectious diseases caused by coronaviruses, e.g., SARS, MERS, and COVID-19.

### Definitions

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, guinea pigs, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”).

As used herein, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, “prophylactic treatment,” “preventive treatment,” “prevent,” “preventing” or “prevention” contemplates an action that occurs before a subject begins to suffer from the specified disease, disorder or condition. In some embodiments, the terms encompass the inhibition or reduction of the seriousness, progression, or recurrence of a symptom of the particular disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier. For example, according to some embodiments, the active ingredient (such as the ACE2 inhibitor or salt thereof, may comprise up to 99% of the composition.

Compound described herein may also comprise one or more isotopic substitutions. For example, H may be in any isotopic form, including 1H, 2H (D or deuterium), and 3H (T or tritium); C may be in any isotopic form, including 12C, 13C, and 14C; O may be in any isotopic form, including 16O and 18O; F may be in any isotopic form, including 18F and 19F; and the like.

The following terms are intended to have the meanings presented therewith below and are useful in understanding the description and intended scope of the present invention. When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example, “C1-6 alkyl” is intended to encompass, C1, C2, C3, C4, C5, C6, C1-6, C1-5, C1-4, C1-3, C1-2, C2-6, C2-5, C2-4, C2-3, C3-6, C3-5, C3-4, C4-6, C4-5, and C5-6 alkyl.

As used herein, “alkyl” refers to a radical of a straight-chain or branched saturated hydrocarbon group, e.g., having 1 to 20 carbon atoms (“C1-20 alkyl”). In some embodiments, an alkyl group has 1 to 10 carbon atoms (“C1-10 alkyl”). In some embodiments, an alkyl group has 1 to 9 carbon atoms (“C1-9 alkyl”). In some embodiments, an alkyl group has 1 to 8 carbon atoms (“C1-8 alkyl”). In some embodiments, an alkyl group has 1 to 7 carbon atoms (“C1-7 alkyl”). In some embodiments, an alkyl group has 1 to 6 carbon atoms (“C1-6 alkyl”). In some embodiments, an alkyl group has 1 to 5 carbon atoms (“C1-5 alkyl”). In some embodiments, an alkyl group has 1 to 4 carbon atoms (“C1-4 alkyl”). In some embodiments, an alkyl group has 1 to 3 carbon atoms (“C1-3 alkyl”). In some embodiments, an alkyl group has 1 to 2 carbon atoms (“C1-2 alkyl”). In some embodiments, an alkyl group has 1 carbon atom (“C1 alkyl”). Examples of C1-6 alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C2-20 alkenyl”). In certain embodiments, alkenyl does not contain any triple bonds. In some embodiments, an alkenyl group has 2 to 10 carbon atoms (“C2-10 alkenyl”). In some embodiments, an alkenyl group has 2 to 9 carbon atoms (“C2-9 alkenyl”). In some embodiments, an alkenyl group has 2 to 8 carbon atoms (“C2-8 alkenyl”). In some embodiments, an alkenyl group has 2 to 7 carbon atoms (“C2-7 alkenyl”). In some embodiments, an alkenyl group has 2 to 6 carbon atoms (“C2-6 alkenyl”). In some embodiments, an alkenyl group has 2 to 5 carbon atoms (“C2-5 alkenyl”). In some embodiments, an alkenyl group has 2 to 4 carbon atoms (“C2-4 alkenyl”). In some embodiments, an alkenyl group has 2 to 3 carbon atoms (“C2-3 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-4 alkenyl groups include ethenyl (C2), 1-propenyl (C3), 2-propenyl (C3), 1-butenyl (C4), 2-butenyl (C4), butadienyl (C4), and the like. Examples of C2-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C2-20 alkynyl”). In certain embodiments, alkynyl does not contain any double bonds. In some embodiments, an alkynyl group has 2 to 10 carbon atoms (“C2-10 alkynyl”). In some embodiments, an alkynyl group has 2 to 9 carbon atoms (“C2-9 alkynyl”). In some embodiments, an alkynyl group has 2 to 8 carbon atoms (“C2-8 alkynyl”). In some embodiments, an alkynyl group has 2 to 7 carbon atoms (“C2-7 alkynyl”). In some embodiments, an alkynyl group has 2 to 6 carbon atoms (“C2-6 alkynyl”). In some embodiments, an alkynyl group has 2 to 5 carbon atoms (“C2-5 alkynyl”). In some embodiments, an alkynyl group has 2 to 4 carbon atoms (“C2-4 alkynyl”). In some embodiments, an alkynyl group has 2 to 3 carbon atoms (“C2-3 alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C2-4 alkynyl groups include, without limitation, ethynyl (C2), 1-propynyl (C3), 2-propynyl (C3), 1-butynyl (C4), 2-butynyl (C4), and the like. Examples of C2-6 alkenyl groups include the aforementioned C2-4 alkynyl groups as well as pentynyl (C5), hexynyl (C6), and the like. Additional examples of alkynyl include heptynyl (C7), octynyl (C8), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

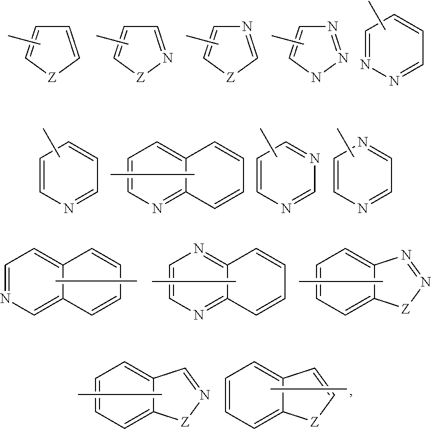

wherein each Z is selected from carbonyl, N, NR65, O, and S; and R65 is independently hydrogen, C1-8 alkyl, C3-10 carbocyclyl, 4-10 membered heterocyclyl, C6-C10 aryl, and 5-10 membered heteroaryl.

As used herein, “carbocyclyl” or “carbocyclic” refers to a radical of a non-aromatic cyclic hydrocarbon group having from 3 to 10 ring carbon atoms (“C3-10 carbocyclyl”) and zero heteroatoms in the non-aromatic ring system. In some embodiments, a carbocyclyl group has 3 to 8 ring carbon atoms (“C3-8 carbocyclyl”). In some embodiments, a carbocyclyl group has 3 to 7 ring carbon atoms (“C3-7 carbocycyl”). In some embodiments, a carbocyclyl group has 3 to 6 ring carbon atoms (“C3-6 carbocyclyl”). In some embodiments, a carbocyclyl group has 5 to 10 ring carbon atoms (“C5-10 carbocyclyl”). Exemplary C3-6 carbocyclyl groups include, without limitation, cyclopropyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 carbocyclyl groups include, without limitation, the aforementioned C3-6 carbocyclyl groups as well as cycloheptyl (C7), cycloheptenyl (C7), cycloheptadienyl (C7), cycloheptatrienyl (C7), cyclooctyl (C8), cyclooctenyl (C8), bicyclo[2.2.1]heptanyl (C7), bicyclo[2.2.2]octanyl (C8), and the like. Exemplary C3-10 carbocyclyl groups include, without limitation, the aforementioned C3-8 carbocyclyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8cycloalkyl,” derived from a cycloalkane. Exemplary cycloalkyl groups include, but are not limited to, cyclohexanes, cyclopentanes, cyclobutanes and cyclopropanes.

As used herein, “C3-6 monocyclic cycloalkyl” or “monocyclic C3-6 cycloalkyl” refers to a 3- to 7-membered monocyclic hydrocarbon ring system that is saturated. 3- to 7-membered monocyclic cycloalkyl groups include, without limitation, cyclopropyl, cyclobutyl, cyclopentyl, and cyclohexyl. Where specified as being optionally substituted or substituted, substituents on a cycloalkyl (e.g., in the case of an optionally substituted cycloalkyl) may be present on any substitutable position and, include, e.g., the position at which the cycloalkyl group is attached.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

In some embodiments, a heterocyclyl group is a 4-7 membered non-aromatic ring system having ring carbon atoms and 1-4 ring heteroatoms, wherein each heteroatom is independently selected from nitrogen, oxygen, and sulfur (“4-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Examples of saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. Where specified as being optionally substituted or substituted, substituents on a heterocyclyl (e.g., in the case of an optionally substituted heterocyclyl) may be present on any substitutable position and, include, e.g., the position at which the heterocyclyl group is attached.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g., heteroaryl; and the like having from 1 to 5, and particularly from 1 to 3 heteroatoms.

As used herein, “cyano” refers to —CN.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “alkoxy,” as used herein, refers to an alkyl group which is attached to another moiety via an oxygen atom (—O(alkyl)). Non-limiting examples include e.g., methoxy, ethoxy, propoxy, and butoxy.

“Haloalkoxy” is a haloalkyl group which is attached to another moiety via an oxygen atom such as, e.g., but are not limited to —OCHCF2 or —OCF3.

The term “haloalkyl” includes mono, poly, and perhaloalkyl groups substituted with one or more halogen atoms where the halogens are independently selected from fluorine, chlorine, bromine, and iodine. For the group C1-4haloalkyl-O—C1-4alkyl, the point of attachment occurs on the alkyl moiety which is halogenated.

As used herein, “nitro” refers to —NO2.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —ORaa, —N(Rcc)2, —CN, —C(═O)Raa, —C(═O)N(Rcc)2, —CO2Raa, —SO2Raa, —C(═NRbb)Raa, —C(═NRcc)ORaa, —C(═NRcc)N(Rcc)2, —SO2N(Rcc)2, —SO2Rcc, —SO2ORcc, —SORaa, —C(═S)N(Rcc)2, —C(═O)SRcc, —C(═S)SRcc, —P(═O)2Raa, —P(═O)(Raa)2, —P(═O)2N(Rcc)2, —P(═O)(NRcc)2, C1-10 alkyl, C1-10 perhaloalkyl, C2-10 alkenyl, C2-10 alkynyl, C3-10 carbocyclyl, 3-14 membered heterocyclyl, C6-14 aryl, and 5-14 membered heteroaryl, or two Rcc groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rdd groups, and wherein Raa, Rbb, Rcc and Rdd are as defined above.

These and other exemplary substituents are described in more detail in the Detailed Description, Examples, and Claims. The invention is not intended to be limited in any manner by the above exemplary listing of substituents.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### Methods of Treatment

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein (e.g., MLN-4760, DX600, ORE-1001, Equivir, Landefill, and other ACE2 inhibitors).

Compounds and compositions described herein are generally useful for inhibiting angiotensin-converting enzyme 2 (ACE2) activity and are useful for treating infection diseases caused by coronavirus, e.g., SARS, MERS, and COVID-19.

In some embodiments, the present invention provides a method of treating or preventing an infectious disease caused by coronavirus using a provided compound. In some embodiments, the present invention provides a method of treating or preventing SARS using a provided compound. In some embodiments, the present invention provides a method of treating or preventing MERS using a provided compound. In some embodiments, the present invention provides a method of treating or preventing COVID-19 using a provided compound.

In some embodiments, the present invention provides a method of treating or preventing an infectious disease caused by coronavirus (e.g., SARS, MERS, and COVID-19) comprising administering to a subject in need thereof a therapeutically effective amount of a compound described herein (e.g., MLN-4760, DX600, ORE-1001, Equivir, Landefill, and other ACE2 inhibitors).

In some embodiments, the present invention provides a method of treating an infectious disease caused by coronavirus in a subject in need thereof, comprising administering to the subject a therapeutically effective amount of an angiotensin-converting enzyme 2 (ACE2) inhibitor, wherein the infectious diseases is selected from SARS, MERS, and COVID-19, and wherein the ACE2 inhibitor is selected from MLN-4760, DX600, ORE-1001, Equivir, and Landefill, and pharmaceutically acceptable salts thereof.

In some embodiments, the present invention provides a method of treating SARS or COVID-19 in a subject in need thereof, comprising administering to the subject a therapeutically effective amount of ACE2 inhibitor, wherein the ACE2 inhibitor is selected from MLN-4760, DX600, ORE-1001, Equivir, and Landefill, and pharmaceutically acceptable salts thereof.

In some embodiments, the present invention provides a method of treating COVID-19 in a subject in need thereof, comprising administering to the subject a therapeutically effective amount of an ACE2 inhibitor, wherein the ACE2 inhibitor is ORE-1001, or a pharmaceutically acceptable salt thereof.

In some embodiments, the present invention provides a method of treating COVID-19 in a subject in need thereof, comprising administering to the subject a therapeutically effective amount of an ACE2 inhibitor, wherein the ACE2 inhibitor is (S,S)-2-[1-carboxy-2-[3-(3,5-dichlorobenzyl)-3H-imidazol-4-yl]-ethylaminol-4-methylpentanoic acid, or a pharmaceutically acceptable salt thereof.

In some embodiments, the present invention provides a method of treating COVID-19 in a subject in need thereof, comprising administering to the subject a therapeutically effective amount of an ACE2 inhibitor, wherein the ACE2 inhibitor is

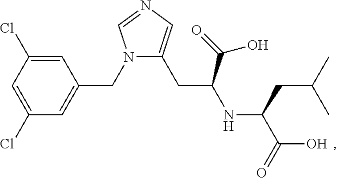 [SMILES: CC(C)C[C@H](N[C@@H](Cc1cncn1Cc1cc(Cl)cc(Cl)c1)C(=O)O)C(=O)O]

or a pharmaceutically acceptable salt thereof.

In some embodiments, present disclosure provides a method of inhibiting transmission of a coronavirus (e.g., SARS-CoV, MERS-Cov, and SARS-CoV-2), comprising administering a therapeutically effective amount of an ACE2 inhibitor (e.g., MLN-4760, DX600, ORE-1001, Equivir, and Landefill), or a pharmaceutically acceptable salt thereof, to a patient suffering from the coronavirus, and/or contacting an effective amount of an ACE2 inhibitor (e.g., MHLN-4760, DX600, ORE-1001, Equivir, and Landefill), or a pharmaceutically acceptable salt thereof, with an infected cell.

In some embodiments, present disclosure provides a method of inhibiting coronavirus (e.g., SARS-CoV, MERS-Cov, and SARS-CoV-2) replication, comprising administering a therapeutically effective amount of an ACE2 inhibitor (e.g., MLN-4760, DX600, ORE-1001, Equivir, and Landefill), or a pharmaceutically acceptable salt thereof, to a patient suffering from the coronavirus, and/or contacting an effective amount of an ACE2 inhibitor (e.g., MLN-4760, DX600, ORE-1001, Equivir, and Landefill), or a pharmaceutically acceptable salt thereof, with an infected cell.

In some embodiments, present disclosure provides a method of minimizing expression of coronavirus (e.g., SARS-CoV, MERS-Cov, and SARS-CoV-2) viral proteins, comprising administering a therapeutically effective amount of an ACE2 inhibitor (e.g., MLN-4760, DX600, ORE-1001, Equivir, and Landefill), or a pharmaceutically acceptable salt thereof, to a patient suffering from the coronavirus, and/or contacting an effective amount of an ACE2 inhibitor (e.g., MLN-4760, DX600, ORE-1001, Equivir, and Landefill), or a pharmaceutically acceptable salt thereof, with an infected cell.

In some embodiments, present disclosure provides a method of inhibiting coronavirus (e.g., SARS-CoV, MERS-Cov, and SARS-CoV-2) release, comprising administering a therapeutically effective amount of an ACE2 inhibitor (e.g., MLN-4760, DX600, ORE-1001, Equivir, and Landefill), or a pharmaceutically acceptable salt thereof, to a patient suffering from the coronavirus, and/or contacting an effective amount of an ACE2 inhibitor (e.g., MLN-4760, DX600, ORE-1001, Equivir, and Landefill), or a pharmaceutically acceptable salt thereof, with an infected cell.

In some embodiments, the methods described herein further comprise identifying a subject having an infectious disease caused by coronavirus (e.g., SARS, MERS, and COVID-19) prior to the administration of a compound described herein.

In some embodiments, the present invention provides the use of a compound described herein (e.g., MLN-4760, DX600, ORE-1001, Equivir, Landefill, and other ACE2 inhibitors) for use in treating an infectious disease caused by coronavirus (e.g., SARS, MERS, and COVID-19) in a subject, wherein the treatment comprises administering a therapeutically effective amount of a provided compound.

In some embodiments, the present invention provides the use of a compound described herein (e.g., MLN-4760, DX600, ORE-1001, Equivir, Landefill, and other ACE2 inhibitors) for use in inhibiting transmission, inhibiting replication, minimizing expression of viral proteins, and/or inhibiting release of a coronavirus (e.g., SARS-CoV, MERS-Cov, and SARS-CoV-2).

In some embodiments, the present invention provides the use of a compound described herein (e.g., MLN-4760, DX600, ORE-1001, Equivir, Landefill, and other ACE2 inhibitors) for the manufacture of a medicament for use in treating an infectious disease caused by coronavirus (e.g., SARS, MERS, and COVID-19) in a subject, wherein the treatment comprises administering a therapeutically effective amount of a provided compound.

In some embodiments, the present invention provides the use of a compound described herein (e.g., MLN-4760, DX600, ORE-1001, Equivir, Landefill, and other ACE2 inhibitors) for the manufacture of a medicament for use in inhibiting transmission, inhibiting replication, minimizing expression of viral proteins, and/or inhibiting release of a coronavirus (e.g., SARS-CoV, MERS-Cov, and SARS-CoV-2).

According to one embodiment, the present method comprises administering to the subject a therapeutically effective amount of a compound selected from the group consisting of ORE-1001 ((S,S)-2-[1-carboxy-2-[3-(3,5-dichlorobenzyl)-3H-imidazol-4-yl]-ethylamino]-4-methylpentanoic acid), pharmaceutically acceptable salts thereof, prodrugs or derivatives thereof.

The chemical structure of ORE-1001 includes two acid moieties. Under suitable conditions, these acid moieties can form salts with suitable bases, and an amino group that, under suitable conditions, can form salts with suitable acids. Internal salts can also be formed. The ORE-1001 compound can be used in its free acid form or in the form of an internal salt, an acid addition salt or a salt with a base. Further compositions useful for inhibiting ACE2 activity and are useful in treating infection diseases caused by coronavirus, e.g., SARS, MERS, and COVID-19 are disclosed below and herein.

According to some embodiments, acid addition salts of ORE-1001 can illustratively be formed with inorganic acids such as mineral acids, for example sulfuric acid, phosphoric acids or hydrohalic (e.g., hydrochloric or hydrobromic) acids; with organic carboxylic acids such as (a) C1-4 alkanecarboxylic acids which may be unsubstituted or substituted (e.g., halo-substituted), for example acetic acid, (b) saturated or unsaturated dicarboxylic acids, for example oxalic, malonic, succinic, maleic, fumaric, phthalic or terephthalic acids, (c) hydroxycarboxylic acids, for example ascorbic, glycolic, lactic, malic, tartaric or citric acids, (d) amino acids, for example aspartic or glutamic acids, or (e) benzoic acid; or with organic sulfonic acids such as C1-4 alkanesulfonic acids or arylsulfonic acids which may be unsubstituted (e.g., halo-substituted), for example methanesulfonic acid or p-toluenesulfonic acid. Exemplary compositions of salts with bases include metal salts such as alkali metal or alkaline earth metal salts, for example sodium, potassium or magnesium salts; or salts with ammonia or an organic amine such as morpholine, thiomorpholine, piperidine, pyrrolidine, a mono-, di- or tri-lower alkyl amine, for example ethylamine, tert-butylamine, diethylamine, diisopropylamine, triethylamine, tributylamine or dimethylpropylamine, or a mono-, di- or tri-(hydroxy lower alkyl) amine, for example monoethanolamine, diethanolamine or triethanolamine.

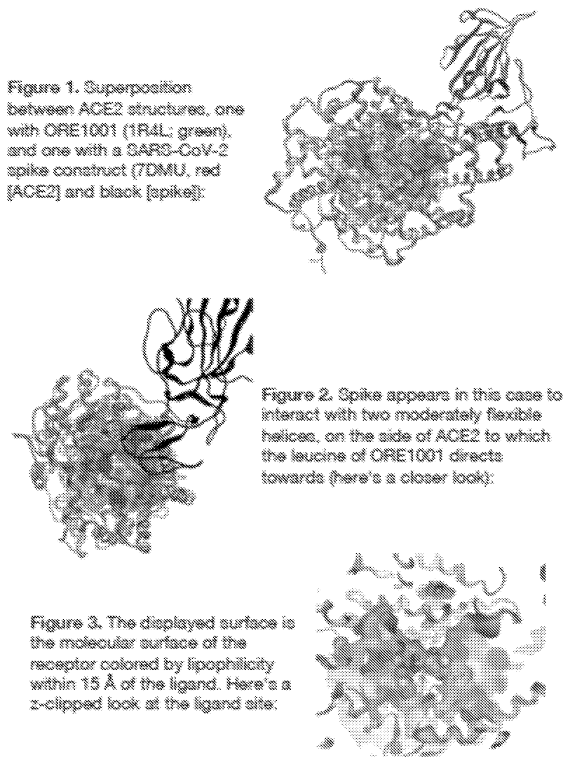

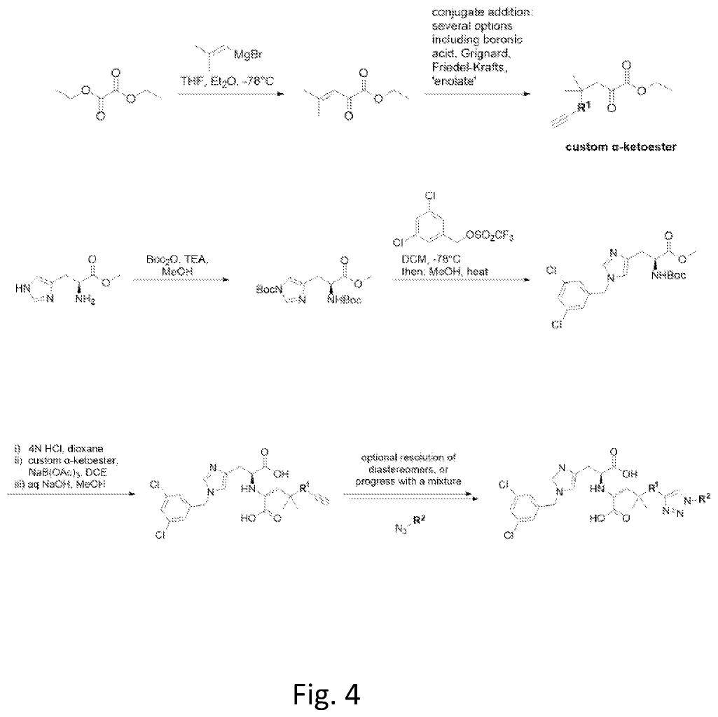

Referring to FIGS. 1-3, illustrations are shown depicting the contact surface of ACE2 structures with ORE-1001 inhibitor and SARS-CoV-2 spike binding. Alignment and superposition were performed with default ‘whole sequence’ settings in MOE (Molecular Operating Environment) software. In particular, referring to FIG. 1, superposition of ACE2 structures with ORE-1001 (PDB ID: 1R4L; green) and with a SARS-CoV-2 spike construct (PDB ID: 7DMU, red [ACE2] and black [spike]) are shown. The SARS-CoV and SARS-CoV-2 utilizes ACE2 as the receptor for viral entry into human cells. The spike (S) protein of SARS-CoV-2, is involved in the receptor recognition and cell membrane fusion process. The spike (S) protein of SARS-CoV-2 is composed of two subunits, S1 and S2. The S1 subunit contains a receptor-binding domain that recognizes and binds to the host receptor angiotensin-converting enzyme 2, while the S2 subunit mediates viral cell membrane fusion (via formation of a six-helical bundle via the two-heptad repeat domain). Referring to FIG. 2, the spike is shown interacting with two moderately flexible helices on the side of ACE2, to which the leucine of ORE-1001 directs towards and that can be used as a suitable linker site to extend out from (to obstruct virus binding). The receptor molecular surface is shown in FIG. 3 to illustrate the ligand site, and the molecular surface of the receptor is depicted colored by lipophilicity (blue: hydrophilic; grey: neutral; green: lipophilic) within 15 Å of the ligand. The superposition of ACE2 structures with ORE-1001 and SARS-CoV-2 spike construct as depicted in FIGS. 1-3, demonstrates a suitable exit vector and linker site to install an appendage to ORE-1001, wherein the ORE-1001 derivative or conjugate could obstruct binding of the virus to angiotensin-converting enzyme 2 (ACE2) without disrupting the preferred bound conformation of the small molecule inhibitor to ACE2. Accordingly, a number of chemical moieties or species, many of which are commercially available, can be appended to generate ORE-1001 conjugates. These can be generated via a minor adaption of the published synthesis route of ORE-1001 found in Dales et al., “Substrate-Based Design Of The First Class Of Angiotensin-Converting Enzyme-Related Carboxypeptidase (ACE2) Inhibitors” JACS, 124 (40), 2002, 11852. FIG. 4 illustrates one such example of an adapted route to allow installing an appendage, which illustrates synthesis of custom α-ketoesters (bearing a terminal alkyne) afforded through a 2-step sequence: a well precedented Grignard addition followed by a slightly less precedented conjugate addition. According to the exemplary description in FIG. 4, R1 and R2 refer to any moiety available through known synthetic methods, or from commercial sources, appropriate for successive steps in synthesis (i.e. no chemoselectivity issues). R1 may be any compact alkyl or (hetero)aryl moiety, and R2 may be any substituent of considerable steric bulk suitable for disruption of the ACE2-spike protein-protein interaction surface (e.g., biotinylated species, as well as fluorescent probes for possible labeling purposes and downstream work). Other suitable processes may be used for preparing the ACE2 inhibitor compounds disclosed herein.

### Compounds

Compounds and compositions described herein are generally useful for inhibiting angiotensin-converting enzyme 2 (ACE2) activity and are useful in treating infection diseases caused by coronavirus, e.g., SARS, MERS, and COVID-19.

In some embodiments, the ACE2 inhibitor of the methods provided herein is a compound selected from MLN-4760, DX600, ORE-1001, Equivir, and Landefill, and a pharmaceutically acceptable salt thereof.

In some embodiments, the ACE2 inhibitor of the methods provided herein is ORE-1001, or a pharmaceutically acceptable salt thereof.

In some embodiments, the ACE2 inhibitor of the methods provided herein is (S,S)-2-[1-carboxy-2-[3-(3,5-dichlorobenzyl)-3H-imidazol-4-yl]-ethylaminol-4-methylpentanoic acid, or a pharmaceutically acceptable salt thereof.

In some embodiments, the ACE2 inhibitor of the methods provided herein is

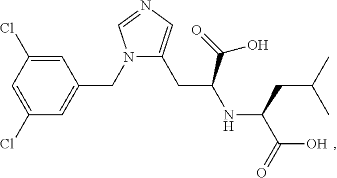 [SMILES: CC(C)C[C@H](N[C@@H](Cc1cncn1Cc1cc(Cl)cc(Cl)c1)C(=O)O)C(=O)O]

or a pharmaceutically acceptable salt thereof.

Combination Therapy

A compound described herein (e.g., MLN-4760, DX600, ORE-1001, Equivir, Landefill, and other ACE2 inhibitors) may be administered in combination with another agent or therapy. A subject to be administered a compound disclosed herein may have a disease, disorder, or condition, or a symptom thereof, that would benefit from treatment with another agent or therapy. These diseases or conditions can relate to an infectious disease caused by coronavirus such as SARS, MERS, and COVID-19.

In some embodiments, the other agent or therapy is an ACE2 inhibitor.

In some embodiments, the other agent or therapy is an anti-viral vaccine, an anti-viral therapeutics, a protease inhibitor, a kinase inhibitor, a fusion inhibitor, a polymerase inhibitor, a neuraminidase inhibitor, a reverse transcriptase inhibitor, or a M2 proton channel inhibitor.

Accordingly, one aspect of the invention provides for a composition comprising the ACE2 inhibitors as described herein and at least one therapeutic agent. In an alternative embodiment, the composition comprises the ACE2 inhibitors as described herein and at least two therapeutic agents. In further alternative embodiments, the composition comprises the ACE2 inhibitors as described herein and at least three therapeutic agents, the ACE2 inhibitors as described herein and at least four therapeutic agents, or the ACE2 inhibitors as described herein and at least five therapeutic agents.

In some embodiments, the methods of combination therapy include co-administration of a single formulation containing the ACE2 inhibitors as described herein and therapeutic agent or agents, essentially contemporaneous administration of more than one formulation comprising the ACE2 inhibitor as described herein and therapeutic agent or agents, and consecutive administration of an ACE2 inhibitor as described herein and therapeutic agent or agents, in any order, wherein preferably there is a time period where the ACE2 inhibitors as described herein and therapeutic agent or agents simultaneously exert their therapeutic effect.

Pharmaceutical Compositions and Routes of Administration

Compounds provided in accordance with the present invention are usually administered in the form of pharmaceutical compositions. This invention therefore provides pharmaceutical compositions that contain, as the active ingredient, one or more of the compounds described herein, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents, such as the agents or therapies described herein. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)).

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection or intravenous infusion. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions or intravenous fluid may be prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprises a disclosed compound, or pharmaceutically acceptable salt thereof, and a pharmaceutically acceptable carrier.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating or preventing an infectious disease caused by coronavirus in a subject in need thereof, comprising administering to the subject a therapeutically effective amount of an angiotensin-converting enzyme 2 (ACE2) inhibitor, wherein the ACE2 inhibitor is selected from MLN-4760, DX600, ORE-1001, Equivir, and Landefill, and pharmaceutically acceptable salts thereof.

2. The method of claim 1, wherein the infectious disease is selected from the group consisting of severe acute respiratory syndrome (SARS), Middle East respiratory syndrome (MERS), and coronavirus disease 19 (COVID-19).

3. The method of claim 1, wherein the infectious disease is SARS.

4. The method of claim 1, wherein the infectious disease is COVID-19.

5. The method of claim 1, wherein the ACE2 inhibitor is ORE-1001 or a pharmaceutical acceptable salt thereof.

6. The method of claim 5, wherein ORE-1001 is (<S′,<S)-2-[1-carboxy-2-[3-(3,5-dichlorobenzyl)-3H-imidazol-4-yl]-ethylaminol-4-methylpentanoic acid, or a pharmaceutically acceptable salt thereof.

7. The method of claim 5, wherein ORE-1001 is, or a pharmaceutically acceptable salt thereof.

8. The method of claim 1, wherein the ACE2 inhibitor is 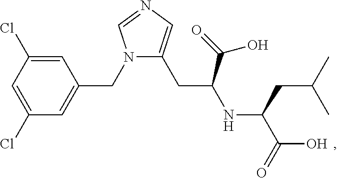 [SMILES: CC(C)C[C@H](N[C@@H](Cc1cncn1Cc1cc(Cl)cc(Cl)c1)C(=O)O)C(=O)O]
or a pharmaceutically acceptable salt thereof.

9. The method of claim 5, wherein said ACE2 inhibitor ORE-1001 or a pharmaceutical acceptable salt thereof obstructs viral binding.

10. The method of claim 9, wherein said ACE2 inhibitor ORE-1001 or a pharmaceutical acceptable salt thereof obstructs the virus' interaction with two moderately flexible helices on the side of ACE2, to which the leucine of ORE-1001 directs towards and that comprises a suitable linker site to extend out from, and thereby to obstruct virus binding.

11. The method of claim 9, wherein said ACE2 inhibitor comprises a pharmaceutically acceptable salt of ORE-1001 that is a fluorophore for labeling and downstream work.

12. The method of claim 5, wherein the ORE-1001 or a pharmaceutical acceptable salt thereof comprises a conjugate that is positioned to block the virus from binding to ACE2.

13. A method of treating or preventing an infectious disease caused by coronavirus in a subject in need thereof, comprising administering to the subject a therapeutically effective amount of an angiotensin-converting enzyme 2 (ACE2) inhibitor, wherein the ACE2 inhibitor is fV,k)-2-[1-carboxy-2-[3-(3,5-dichlorobenzyl)-3H-imidazol-4-yl]-ethylaminol-4-methylpentanoic acid, or a pharmaceutically acceptable salt thereof, and wherein the (S,S)˜2-[1-carboxy-2-[3-(3,5-dichlorobenzyl)-3H-imidazol-4-yl]-ethylaminol-4-methylpentanoic acid, or a pharmaceutically acceptable salt thereof comprises a conjugate that is positioned to block the virus from binding to ACE2.

14. The method of claim 2, wherein the ACE2 inhibitor is ORE-1001 or a pharmaceutical acceptable salt thereof.

15. The method of claim 3, wherein the ACE2 inhibitor is ORE-1001 or a pharmaceutical acceptable salt thereof.

16. The method of claim 4, wherein the ACE2 inhibitor is ORE-1001 or a pharmaceutical acceptable salt thereof.

17. The method of claim 2, wherein the ACE2 inhibitor is 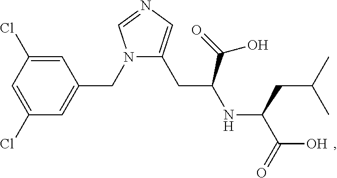 [SMILES: CC(C)C[C@H](N[C@@H](Cc1cncn1Cc1cc(Cl)cc(Cl)c1)C(=O)O)C(=O)O]
or a pharmaceutically acceptable salt thereof.

18. The method of claim 3, wherein the ACE2 inhibitor is 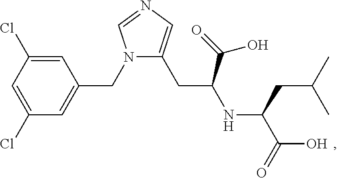 [SMILES: CC(C)C[C@H](N[C@@H](Cc1cncn1Cc1cc(Cl)cc(Cl)c1)C(=O)O)C(=O)O]
or a pharmaceutically acceptable salt thereof.

19. The method of claim 3, wherein the ACE2 inhibitor is 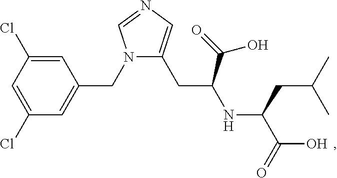 [SMILES: CC(C)C[C@H](N[C@@H](Cc1cncn1Cc1cc(Cl)cc(Cl)c1)C(=O)O)C(=O)O]
or a pharmaceutically acceptable salt thereof.

20. The method of claim 4, wherein the ACE2 inhibitor is 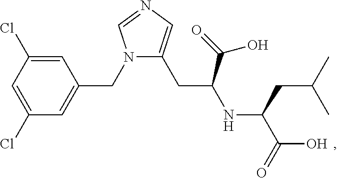 [SMILES: CC(C)C[C@H](N[C@@H](Cc1cncn1Cc1cc(Cl)cc(Cl)c1)C(=O)O)C(=O)O]
or a pharmaceutically acceptable salt thereof.
